(19) United States
(12) Patent Application Publication
(10) Pub. No.: US 2024/0203606 A1
(43) Pub. Date: Jun. 20, 2024

(54) Methods for Providing Virtual Interactive Therapy Sessions

(71) Applicant: Alejandra Cardenas Oliveros, Chicago, IL (US)

(72) Inventor: Alejandra Cardenas Oliveros, Chicago, IL (US)

(21) Appl. No.: 18/082,603

(22) Filed: Dec. 16, 2022

(51) Int. Cl. G16H 80/00; G16H 20/70

(52) U.S. Cl. CPC G16H 80/00; G16H 20/70

(57) ABSTRACT

The present disclosure provides a computer-implemented process for aiding a child therapy session, where a number of pre-generated virtual therapy rooms with a patient avatar and a therapist avatar inserted are displayed to a patient device and a therapist device simultaneously during a remote therapy session. Each therapy room display comprises multimedia objects and interactive elements for assisting the therapist in bringing a child through a therapy session in an enjoyable manner, and the rooms are linked so as to be displayed in a helpful sequence which can be tailored to the content of the therapy session. The disclosed method thus provides a low cost and completely adaptable support tool for remote therapy.

### FIELD OF INVENTION

The present invention relates generally to computer-implemented therapy aids. More specifically, the present invention relates to a method for providing a virtual interactive therapy room for aiding children's therapy sessions.

### BACKGROUND

The importance of mental health issues has become more and more evident in recent years, with a much more widespread societal acceptance of counseling and therapy services, including to children.

Providing counseling and therapy services to children often encompasses novel and effective types of pediatric counseling techniques, such as play therapy. Present play therapy centers are implanted in fixed places to which the parents must go with their child, which is sometimes inconvenient, uncomfortable, and even difficult, depending on the distance from their home.

In many under-served communities, individuals and families struggle to obtain access to therapy systems due to logistics and the expensive nature of the services, resulting in years of unaddressed pain and mental health issues, particularly for those who face barriers to transportation.

Furthermore, recent research demonstrates that when mobile therapy services are provided in the form of a counselor coming to the patient's home and providing services therein, the results have mixed success due to family distractions, uncomfortable atmospheres, and invasions of privacy and confidentiality for the patient and the counselor.

There exists a need in such communities, and the world over, for the ability to receive mental health therapy and/or counseling services for children that are provided in a manner which does not require travel by either the therapist or the child and their parents, and which is cost-effective to implement and adaptable to different clients and therapeutic styles.

It is within this context that the present invention is provided.

### SUMMARY

The present disclosure provides a computer-implemented process for aiding a child therapy session, where a number of pre-generated virtual therapy rooms with a patient avatar and a therapist avatar inserted are displayed to a patient device and a therapist device simultaneously during a remote therapy session. Each therapy room display comprises multimedia objects and interactive elements for assisting the therapist in bringing a child through a therapy session in an enjoyable manner, and the rooms are linked so as to be displayed in a helpful sequence which can be tailored to the content of the therapy session. The disclosed method thus provides a low cost and completely adaptable support tool for remote therapy.

According to a first aspect of the present disclosure, there is provided a computer-implemented method for aiding a remote therapy session, the method comprising the steps of: generating a therapist avatar; generating a patient avatar; generating a plurality of virtual therapy room displays, each therapy room display having a predefined purpose and comprising one or more interactive elements and/or a multimedia therapy aid; placing the therapist avatar and the patient avatar in a number of the virtual therapy room displays, the placement and stance of the avatars in each room display illustrating or aiding the predefined purpose of the room in coordination with the one or more interactive elements and/or a multimedia therapy aids; and displaying one or more of the virtual therapy rooms to a therapist device and a patient device simultaneously during a remote therapy session; and changing which room is displayed based on input received from the patient device or the therapist device.

In some embodiments, the virtual therapy room displays comprise a welcome room with a consistent design to create a safe and calming space for the patient.

The welcome room may comprises links to one or more informative rooms with therapy aids for explaining therapeutic boundaries and parental roles.

In some embodiments, the virtual therapy room displays comprise one or more rooms with infographic therapy aids for emotional education.

In some embodiments, the virtual therapy room displays comprise one or more rooms with infographic therapy aids for outlining a therapy schedule.

In some embodiments, the virtual therapy room displays comprise a room with an infographic therapy aid for assisting in determining a current emotional state of the patient.

The emotional state room may comprises links to a plurality of other therapy room displays, each linked room comprising a therapy aid infographic or interactive element associated with a selected emotional state.

In such examples, the linked rooms comprise a room with a plurality of games, books, and educational resources for educating the patient about emotions related to sadness; a room with a plurality of games, books, and educational resources for educating the patient about emotions related to anger; a room with a plurality of games, books, and educational resources for educating the patient about emotions related to anxiety; or a room with a plurality of games, books, and educational resources for educating the patient about emotions related to happiness.

Furthermore, one or more of the linked rooms may in turn be linked to a room comprising multimedia including music and games designed for sensory stimulation.

In some embodiments, one or more of the virtual therapy room displays comprise a collection of educational content designed to teach a patient about a specific developmental topic.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The terminology used herein is for the purpose of describing particular embodiments only and is not intended to be limiting. As used herein, the singular forms “a,” “an” and “the” are intended to include the plural forms as well, unless the context clearly indicates otherwise.

The terms “patient,” may be used herein interchangeably with the words “client,” “employee,” “recipient,” etc. Such terms are intended to be used synonymously and refer to a user, group of users, business, and/or other entity seeking counseling or advice via the present invention.

The term “therapist,” as used herein may be used interchangeably with the terms “expert,” “provider,” “physician,” “practitioner,” “contractor,” “employer,” “professional,” etc. Such terms are intended to be used synonymously and refer to an individual, a group of individuals, a business, and/or other entity that provides counseling via the present invention.

The present disclosure relates to a computer-implemented method for assisting with a virtual or remote therapy session carried out over a communications network via two multimedia-enabled user devices. Generally, each user device is in communication with the network through a communications channel. The network is also in communication with server and may further communicate with a database.

The user devices may include conventional software such as productivity tools (e.g., word processing, spreadsheets, etc.) at least one browser, and video communications technology such as Zoom, Microsoft Teams, Google Meets, etc.

The method for aiding a remote therapy session includes the steps of generating a therapist avatar and generating a patient avatar. These may be, for example, cartoon representations of a therapist and a child patient, capable of being illustrated in various stances and with various expressions.

The method further comprises generating a plurality of virtual therapy room displays, each therapy room display having a predefined purpose and comprising one or more interactive elements and/or a multimedia therapy aid.

The therapy aids may be infographics displaying educational or assistive content such as an emotional chart or a therapy schedule.

The interactive elements may be links to various resources, including links to other therapy rooms, games, music, and books or articles. Clicking on an interactive element by either the therapist device or the patient device may cause both devices to access said resources—for example causing a piece of music to play.

The therapy room displays, which may be actual virtual therapy rooms managed by the server or may simply be slides with visual representations of therapy rooms, each have a therapist avatar placed in them which is interacting with the therapy aid objects, and one or more of the rooms may also have the patient avatar placed in it and interacting with either the therapist avatar or the objects in the room display.

The virtual therapy rooms are displayed in a certain predefined sequence that is intended to accompany the steps of a therapy session. This sequence can be altered and tailored beforehand according to a therapist's desires, but in general it will always start with a welcome room with a consistent design to create a safe and calming space for the child, so that they can relax into the session and feel comfortable. The welcome room may have links to resources such as a set of confidentiality rules, or to guidelines about parental input.

The displayed room will change for both devices based on input from the user devices, for example clicking an element for the welcome room may cause the display to shift to a first educational content room, or to a room for assessing the child's current emotional state.

The emotional state of the child may be divided into a number of categories, with links to different rooms for dealing with and educating the child on each category. In such examples, the linked rooms may comprise a room with a plurality of games, books, and educational resources for educating the patient about emotions related to, respectively: sadness, anger, anxiety/frustration, happiness, and other emotions.

The rooms for each emotional state may have appropriate colour schemes and designs for synchronising with the child's mood, as well as appropriate multimedia such as sad music, calming activity games, etc, and related educational resources and articles.

For when the child is overly energetic, the virtual rooms may also comprise multimedia such as dance music and intense games to help the child burn off their excess energy.

The invention relates generally to methods and systems for conducting counselling sessions. More particularly, the invention relates to methods and systems for counselling sessions and clinics conducted remotely, such as over the Internet and/or via satellite using WIFI as well as other web-based technologies. In addition, the invention relates to methods and systems for conducting remote counselling sessions by means of various mobile devices.

The disclosed method can be run by any suitable type of computer. A computer may be a uniprocessor or multiprocessor machine. Accordingly, a computer may include one or more processors and, thus, the aforementioned computer system may also include one or more processors. Examples of processors include sequential state machines, microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), gated logic, programmable control boards (PCBs), and other suitable hardware configured to perform the various functionality described throughout this disclosure.

Additionally, the computer may include one or more memories. Accordingly, the aforementioned computer systems may include one or more memories. A memory may include a memory storage device or an addressable storage medium which may include, by way of example, random access memory (RAM), static random access memory (SRAM), dynamic random access memory (DRAM), electronically erasable programmable read-only memory (EEPROM), programmable read-only memory (PROM), erasable programmable read-only memory (EPROM), hard disks, floppy disks, laser disk players, digital video disks, compact disks, video tapes, audio tapes, magnetic recording tracks, magnetic tunnel junction (MTJ) memory, optical memory storage, quantum mechanical storage, electronic networks, and/or other devices or technologies used to store electronic content such as programs and data. In particular, the one or more memories may store computer executable instructions that, when executed by the one or more processors, cause the one or more processors to implement the procedures and techniques described herein. The one or more processors may be operably associated with the one or more memories so that the computer executable instructions can be provided to the one or more processors for execution. For example, the one or more processors may be operably associated to the one or more memories through one or more buses. Furthermore, the computer may possess or may be operably associated with input devices (e.g., a keyboard, a keypad, controller, a mouse, a microphone, a touch screen, a sensor) and output devices such as (e.g., a computer screen, printer, or a speaker).

The computer may execute an appropriate operating system such as LINUX®, UNIX®, MICROSOFT® WINDOWS®, APPLE® MACOS®, IBM® OS/2º, Digital Multiplex Signal DMX 512, CHROME® OS, ANDROID, and PALM® OS, and/or the like. The computer may advantageously be equipped with a network communication device such as a network interface card, a modem, or other network connection device suitable for connecting to one or more networks.

A computer may advantageously contain control logic, or program logic, or other substrate configuration representing data and instructions, which cause the computer to operate in a specific and predefined manner as, described herein. In particular, the computer programs, when executed, enable a control processor to perform and/or cause the performance of features of the present disclosure. The control logic may advantageously be implemented as one or more modules. The modules may advantageously be configured to reside on the computer memory and execute on the one or more processors. The modules include, but are not limited to, software or hardware components that perform certain tasks. Thus, a module may include, by way of example, components, such as, software components, processes, functions, subroutines, procedures, attributes, class components, task components, object-oriented software components, segments of program code, drivers, firmware, micro code, circuitry, data, and/or the like.

The control logic conventionally includes the manipulation of digital bits by the processor and the maintenance of these bits within memory storage devices resident in one or more of the memory storage devices. Such memory storage devices may impose a physical organization upon the collection of stored data bits, which are generally stored by specific electrical or magnetic storage cells.

The control logic generally performs a sequence of computer-executed steps. These steps generally require manipulations of physical quantities. Usually, although not necessarily, these quantities take the form of electrical, magnetic, or optical signals capable of being stored, transferred, combined, compared, or otherwise manipulated. It is conventional for those skilled in the art to refer to these signals as bits, values, elements, symbols, characters, text, terms, numbers, files, or the like. It should be kept in mind, however, that these and some other terms should be associated with appropriate physical quantities for computer operations, and that these terms are merely conventional labels applied to physical quantities that exist within and during operation of the computer based on designed relationships between these physical quantities and the symbolic values they represent.

It should be understood that manipulations within the computer are often referred to in terms of adding, comparing, moving, searching, or the like, which are often associated with manual operations performed by a human operator. It is to be understood that no involvement of the human operator may be necessary, or even desirable. The operations described herein are machine operations performed in conjunction with the human operator or user that interacts with the computer or computers.

It should also be understood that the programs, modules, processes, methods, and the like, described herein are but an exemplary implementation and are not related, or limited, to any particular computer, apparatus, or computer language. Rather, various types of general purpose computing machines or devices may be used with programs constructed in accordance with some of the teachings described herein. In some embodiments, very specific computing machines, with specific functionality, may be required. Similarly, it may prove advantageous to construct a specialized apparatus to perform the method steps described herein by way of dedicated computer systems with hard-wired logic or programs stored in nonvolatile memory, such as, by way of example, read-only memory (ROM).

In some embodiments, features of the computer systems can be implemented primarily in hardware using, for example, hardware components such as application specific integrated circuits (ASICs) or field-programmable gated arrays (FPGAs). Implementation of the hardware circuitry will be apparent to persons skilled in the relevant art(s). In yet another embodiment, features of the computer systems can be implemented using a combination of both general—purpose hardware and software

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the method have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

2. The vacuum system having an independent inlet condenser set and a root vacuum pump set as claimed in claim 1, wherein the independent inlet condenser set (10) is connected to a liquid collecting and recycling tank unit (20) for collecting condensed liquid and soft water from the independent inlet condenser set (10), in that, the soft water is from the air cooling power generator condenser (100).

3. The vacuum system having a condenser and a root vacuum pump set as claimed in claim 1, wherein the liquid collecting and recycling tank unit (20) is capable to collect condensed liquid and soft water in vacuum state automatically and continuously.

4. The vacuum system having a condenser and a root vacuum pump set as claimed in claim 1, wherein the inlet end (11) of the independent inlet condenser set (10) is capable of suffering from a pressure larger than 10000 Pa.

5. The vacuum system having a condenser and a root vacuum pump set as claimed in claim 1, further comprising: a heat exchanger (60) serially connected to the output pipe (33) for cooling air outputted from the root vacuum pump set (30) and the cooled air being inputted to the backing pump (50).

6. The vacuum system having a condenser and a root vacuum pump set as claimed in claim 1, wherein the root vacuum pump (40) is capable of suffering from a pressure larger than 10000 Pa (Pascal).

7. The vacuum system having a condenser and a root vacuum pump set as claimed in claim 1, wherein the root vacuum pump (40) is an air cooling root vacuum pump.

8. The vacuum system having a condenser and a root vacuum pump set as claimed in claim 1, wherein the root vacuum pump (40) is connected to an electric driving device (405) for driving a respective root vacuum pump.

9. (canceled)

10. (canceled)

11. The vacuum system having a condenser and a root vacuum pump set as claimed in claim 1, wherein output air of the backing pump (50) is drained out to the atmosphere.

12. The vacuum system having a condenser and a root vacuum pump set as claimed in claim 1, wherein the backing pump (50) is a liquid circulated pump.

13. The vacuum system having a condenser and a root vacuum pump set as claimed in claim 1, wherein when the air pressure of the air from the root vacuum pump (40) of the root vacuum pump set (30) is lower than a predetermined value, the backing pump (50) do not acted, while the air from the root vacuum pump set (30) directly bypasses through the backing pump (50) to be drained out.

14. The vacuum system having a condenser and a root vacuum pump set as claimed in claim 1, wherein liquid collecting and recycling tank unit (20) has an automatic double valve set up device (201) and a water level gauge (202) that is connected to a controller (203).

15. The vacuum system having a condenser and a root vacuum pump set as claimed in claim 1, wherein the root vacuum pump (40) has a casing (41) which has an inlet (411) and an outlet (412); an interior of the casing (41) is formed with a vacuum chamber (42) and two bearing rooms (43) at two sides of the vacuum chamber (42); the vacuum chamber (42) is connected and communicated to the inlet (411) and the outlet (412); a driving shaft (44) is installed within the casing (41) and penetrates through the vacuum chamber (42) and the two bearing rooms (43); one end of the driving shaft (44) passes out of a right wall (415) of the casing (41); a blade set (45) is installed within the vacuum chamber (42) and is installed on the driving shaft (44); the gas mixture is inputted from the inlet (411) to the vacuum chamber (42); by rotation of the blade set (45), the gas mixture is compressed and is outputted from the outlet (412); inner connection walls (413), (414) between the vacuum chamber (42) and the two bearing rooms (43) are installed with respective bearings (46) which are arranged to be around the driving shaft (44); as well as an opening of the right wall (415) of the casing (41) is formed with another bearing (46) around the driving shaft (44); the bearings (46) support the driving shaft (44); the bearings (46) completely seal spaces between the driving shaft (44) and the inner walls of the casing (41) so that the vacuum chamber (42) is completely isolated from the two bearing rooms (43); therefore, liquid out of the casing (41) and in the two bearing rooms (43) cannot permeate into the vacuum chamber (42); furthermore, the gas mixture in the vacuum chamber (42) cannot enter into the bearing rooms (43); therefore, in operation, interior of the vacuum chamber (42) of the root vacuum pump (40) only has original air and the gas mixture without any impurities; moreover, liquid within the bearing rooms (43) cannot drain out of the casing (41).

16. The vacuum system having a condenser and a root vacuum pump set as claimed in claim 15, wherein the root vacuum pump (40) has a structure which can suffer a great pressure difference; the great pressure means that the root vacuum pump (40) can operate under an inlet pressure of 5000 Pa to 30000 Pa in a whole day under a condition that the condenser is in a vacuum state and can suffer from a pressure difference larger than 5000 Pa.

17. The vacuum system having a condenser and root vacuum pump set as claimed in claim 15, wherein the root vacuum pump (40) operates under temperatures larger than 130° C.
